(19) United States
(12) Patent Application Publication
(10) Pub. No.: US 2013/0217957 A1
(43) Pub. Date: Aug. 22, 2013

(54) DEVICES AND METHODS FOR THE ENDOLUMENAL TREATMENT OF OBESITY

(71) Applicants: Tracy D. Maahs, Yorba Linda, CA (US); John A. Cox, San Clemente, CA (US); Christopher James Earley, San Clemente, CA (US)

(72) Inventors: Tracy D. Maahs, Yorba Linda, CA (US); John A. Cox, San Clemente, CA (US); Christopher James Earley, San Clemente, CA (US)

(73) Assignee: USGI Medical, Inc., San Clemente, CA (US)

(21) Appl. No.: 13/760,874

(22) Filed: Feb. 6, 2013

Related U.S. Application Data
(60) Provisional application No. 61/599,804, filed on Feb. 16, 2012.

(51) Int. Cl. A61B 17/12

(52) U.S. Cl. CPC A61B 17/12013
USPC 600/37

(57) ABSTRACT

A surgical method for treating obesity by reducing the size and/or function of the stomach includes forming at least two plications or tissue folds in tissue of a patient using anchor assemblies having a loop. The plications are then optionally allowed to heal. A loop suture or wire is threaded through the loops. The loop suture is then tensioned to draw the plications towards each other. The loop suture is then secured via a knot or a cinch. When the method is performed in the stomach for treatment of obesity, forming the plications reduces the volume of the stomach. Drawing the plications together creates a contracted tissue area which further reduces the volume of the stomach. Additional plications may also be formed and drawn together with the same loop suture, or with a different loop suture.

This patent application claims priority to U.S. Provisional Patent Application No. 61/599,804, filed Feb. 16, 2012, incorporated herein by reference.

### BACKGROUND OF THE INVENTION

Obesity is a serious healthcare problem in the United States, and increasingly, in developing countries as well. The National Institutes of Health (NIH) estimate that about two-thirds of adults—133.6 million people—in the United States are overweight or obese, while almost 5% of adults—15 million Americans—are considered extremely obese. Obese adults are at increased risk of type II diabetes, hypertension, stroke, certain cancers, and other serious health conditions. The annual medical costs of obesity in the United States are estimated to be over $100 billion.

As obesity rates continue to rise, patients have sought both surgical and non-surgical weight loss options. Surgical procedures for treating obesity, known as bariatric surgery, reduce the size of the stomach and/or change the anatomy of the upper gut, resulting in weight loss. An especially promising type of bariatric surgery is incisionless endolumenal stomach surgery. In one form of endolumenal stomach surgery, a long flexible tool is routed through the mouth and throat and into the patient's stomach. The tool is then used by the surgeon to make substantially permanent tissue folds or plications in the stomach tissue, which reduces the size and changes the function of the stomach. Examples of this type of endolumenal surgery are described in United States Patent Application Publication No. 2009/0255544, incorporated herein by reference. Endolumenal techniques differ from traditional surgery in the access method used to complete the procedure. Endolumenal procedures use the natural orifices (e.g. the mouth) to access the stomach, eliminating the need for the physician to cut through the abdominal wall. Advantages of endolumenal techniques include reduced risk of wound infection, hernias, and adhesions, and they offer the patient less post-operative pain, faster recovery time, and no abdominal scars. While these and other techniques have worked well in many surgical patients, disadvantages remain and improved methods and devices are needed.

### SUMMARY OF THE INVENTION

An improved surgical method includes forming at least two plications or tissue folds in tissue of a patient using anchor assemblies having a loop. The plications are then allowed to heal or are designed to be retained during the healing process. A loop suture, wire or zip tie is threaded through the loops. The loop suture is then tensioned to draw the plications towards each other. The loop suture is then secured via a knot or a cinch. The method may be performed in the stomach for treatment of obesity, with the plications reducing the volume of the stomach and/or changing the fundamental function of the stomach as a reservoir and emulsifier of food. Drawing the plications together creates a contracted tissue area which further reduces the volume of the stomach. In addition, this looping of plications may be used to force the food to empty into the small intestine at a different rate than before the procedure, and it may be used to force the early firing of stretch receptors in the stomach. Additionally, the process may be used to prevent the firing of these receptors or to change the timing or quantity of the release of special gut hormones known to effect hunger, satiety, eating behavior, or nervous system and autonomic functions. Additional plications may also be formed and drawn together with the same loop suture, or with a different loop suture.

The method optionally includes bringing the first and second ends of the loop suture out of the patient, providing a knot or a cinch on the first and second ends of the loop suture, and pushing the knot or cinch along the suture to a position adjacent to at least one of the first and second plications.

A surgical system includes an instrument for forming plications, and one or more instruments for threading a loop suture, wire or zip tie through the loops at the plications. The instruments may optionally be adapted for withdrawing both ends of the loop suture out of the patient's body, for placement of a knot or cinch on the ends of the loop suture. The present methods may be performed endolumenally or laparoscopically, or using a combination of both techniques.

Other and further objects and advantages will be apparent from the following detailed description, which provides various embodiments and examples. The invention resides as well in sub-combinations of the devices and methods described.

### BRIEF DESCRIPTION OF THE DRAWINGS

In the drawings, the same reference number indicates the same element in each of the views.

FIG. 1 is an illustration of an endolumenal access system and tissue anchor delivery device advanced endolumenally into a stomach.

FIG. 2 is an exploded view of a tissue anchor assembly delivery device.

FIG. 3 is an exploded view of a needle deployment assembly.

FIGS. 4 through 6 are side views of a tissue manipulation assembly and helical tissue engagement instrument engaging stomach tissue.

FIGS. 7 through 12 are illustrations showing a progression of an endolumenal obesity treatment procedure.

FIG. 13 is a schematic representation of a tissue anchor assembly.

FIG. 14 is a schematic representation of a tissue anchor assembly securing a tissue fold.

FIG. 15-17 are diagrams illustrating surgical methods for forming contracted tissue areas.

FIGS. 18-23 are enlarged views showing plications being drawn together and secured.

FIG. 24 is a diagram of an alternative anchor assembly.

FIGS. 25-29 show a method using a loop suture having loop end interlocked with a loop on a plication.

FIGS. 30-31 show alternative anchor assemblies for forming plications having a loop.

FIGS. 32-34 show an example of a cinch.

FIG. 35 illustrates a loop suture having a threading on stiffening element at the distal end.

FIGS. 36-39 show a sequence of operation of a loop threading tool.

FIGS. 40-43 show a sequence of operation of an alternative loop threading tool.

FIGS. 44-46 show a sequence of operation of another loop threading tool.

FIGS. 47-49 show a sequence of operation of a loop threading tool.

FIGS. 50 and 51 show an example of a cinch for securing both ends of a loop suture.

FIG. 52 shows a modified cinch including an eyelet.

FIGS. 53-55 show a design for a knot pusher.

FIGS. 56-57 show a slip knot and cinch combination.

### DETAILED DESCRIPTION

The methods and devices described here are especially useful for stomach reduction and/or alteration surgery, for the treatment of obesity. However, these methods and devices may also be used for other surgical procedures, and to treat other conditions as well.

As described in United States Patent Application Publication No. 2009/0255544, incorporated herein by reference, obesity may be treated by forming substantially permanent tissue folds or plications in stomach tissue. The stomach has unique characteristics affecting several aspects of stomach surgery. The stomach has four tissue layers. The mucosa layer is the inner tissue layer followed by connective tissue, the muscularis layer and the serosa layer. The muscularis and serosal layers are difficult to manipulate because they are only loosely adhered to the mucosal layer and resistant to surgical modification.

The mucosa and connective tissue layers typically do not heal together in a way that can sustain the tensile loads imposed by normal movement of the stomach wall during ingestion and processing of food. Forming mucosa-to-mucosa tissue folds will not result in permanent plication formation. Rather, the fold must involve deeper tissue layers. Forming tissue folds in the sub-mucosa layer and muscularis layer may result in permanent tissue remodeling. Forming tissue folds with serosa to serosa contact will generally consistently result in the tissue fold healing together to form a permanent plication.

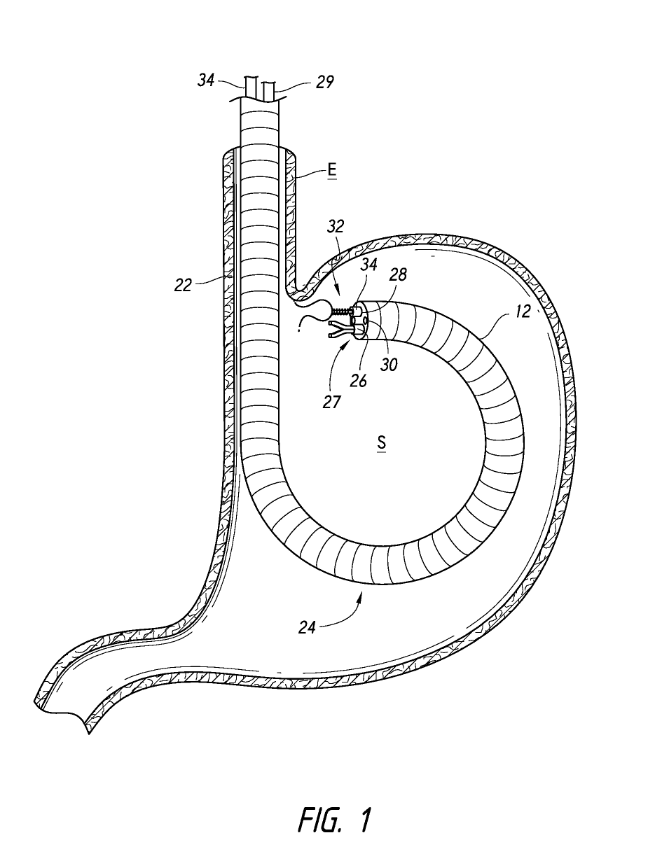

FIGS. 1-12 generally illustrate endolumenal instruments and methods for forming plications in stomach tissue. As illustrated in FIG. 1, an endoscopic system 12 is advanced into a patient's stomach S per-orally and through the esophagus E. The endoscopic system 12 includes an endoscopic device having a distal portion that is articulated and steered to position its distal end at a desired location within the stomach S. The endoscopic system 12 is a flexible, steerable tube having lumens, and optionally having the capability of shape-locking, to allow for procedures to be performed on the tissue utilizing any number of tools delivered through the system 12.

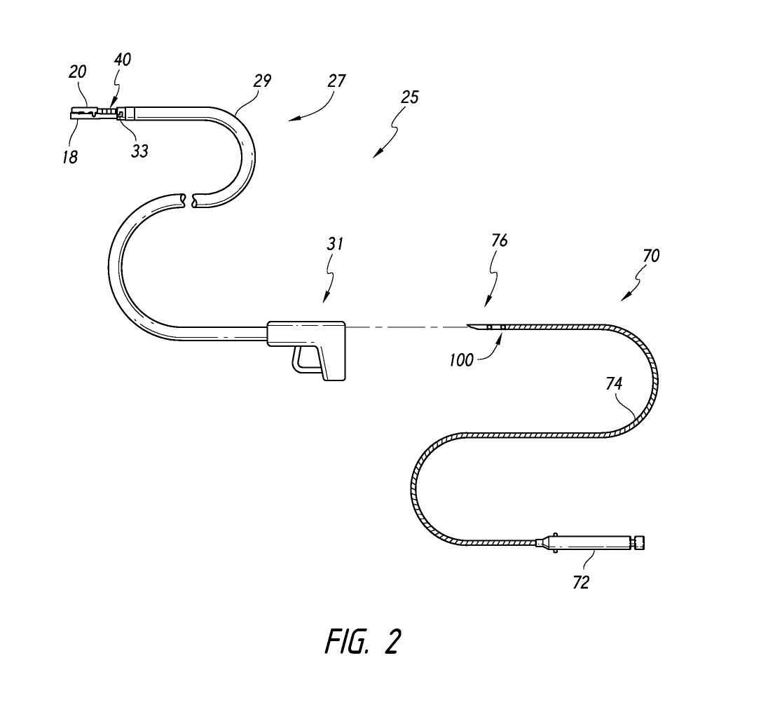

Referring still to FIG. 1, endoscopic system 12 has a steerable end 24. The endoscopic system 12 may have at least first and second lumens 26, 28, respectively, through which one or more tools, such as the endolumenal tool 25 shown in FIG. 2 may be deployed into the stomach S. Additional lumens may be provided through the endoscopic system 12, such as a visualization lumen 30, through which an endoscope may be positioned to provide visualization of the region of tissue. Alternatively, an imager such as a CCD imager or optical fibers may be provided in lumen 30 to provide visualization. An optional thin wall sheath may be disposed through the patient's mouth, esophagus E, and possibly past the gastroesophageal junction into the stomach S. The endoscopic system 12 may be advanced through the esophagus E and into the stomach S while in a flexible state.

As shown in FIG. 2, an endolumenal tool 25 includes a tissue manipulation assembly 27 and an anchor deployment assembly 70. The tissue manipulation assembly 27 includes a tubular body 29 which is flexible so that it can be readily advanced into a body lumen, e.g., transorally, percutaneously, laparoscopically, etc. The tubular body 29 can transmit torque or angular twisting movement, so that when a handle 31 is rotated by the surgeon from outside the patient's body, the distal end of tubular body 29 rotates in a corresponding manner. FIG. 1 shows the tissue manipulation assembly 27 advanced through the first lumen 26 and a helical tissue engagement member 32 positioned on a flexible shaft 34 advanced through the second lumen 28.

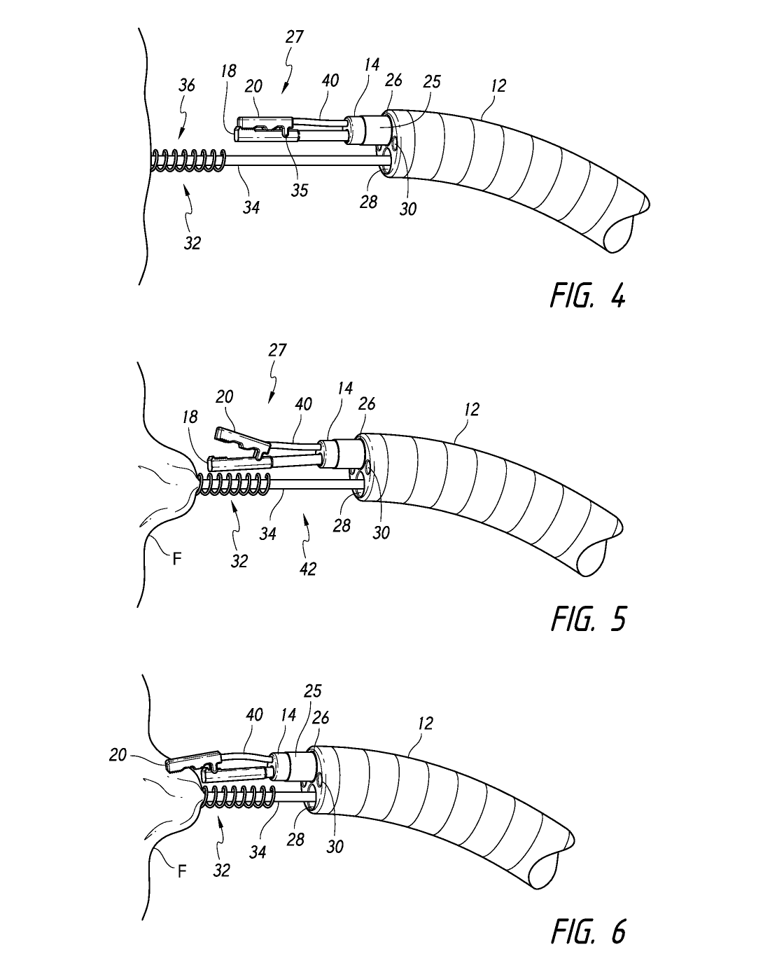

As shown in FIGS. 2 and 5, the tissue manipulation assembly 27 includes a pair of jaws at the distal end of the tubular body 29, and optionally attached to the distal end of the tubular body via a pivotable coupling 33. A lower jaw 18 extends forward from the pivotable coupling 33 and an upper jaw 20, in this example, is pivotably coupled to the lower jaw via a jaw pivot 35 shown in FIG. 4. The location of the jaw pivot may be positioned at various locations along the lower jaw 18 depending upon a number of factors, e.g., the desired size of the “bite” or opening for accepting tissue between the jaw members, the amount of closing force between the jaws, etc. One or both jaws may also have a number of protrusions, projections, grasping teeth, textured surfaces.

A launch tube 40 extends from the handle 31, through the tubular body 29, with a distal end of the launch tube 40 pivotally connected to the upper jaw 20 at a launch tube pivot. A distal portion of the launch tube 40 may be pivoted into position within a channel or groove in upper jaw 20, to provide a low-profile configuration of the tissue manipulation assembly 27. When articulated, either via the launch tube 40 or other mechanism, the jaws may be urged into an open configuration to receive and grasp tissue. When the launch tube is advanced, using a control at the handle 31, the launch tube 40 may bend into a curved shape that positions the launch tube opening substantially perpendicular to the upper jaw 20. The launch tube 40, or at least the exposed portion of the launch tube 40, may be fabricated from a highly flexible material or it may be fabricated, e.g., from Nitinol tubing material which is adapted to flex, e.g., via circumferential slots, to permit bending.

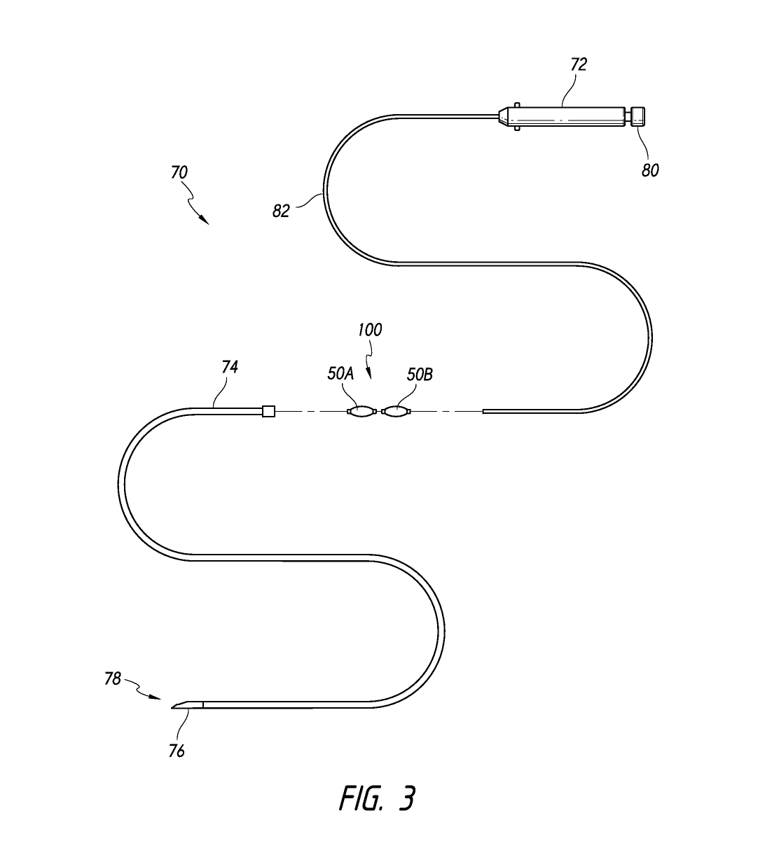

As shown in FIGS. 2 and 3, an anchor deployment assembly 70 may be deployed through the tissue manipulation assembly 27, through the handle 31 and through the tubular body 29. Referring to FIG. 3, the anchor deployment assembly 70 includes a needle 76 at the distal tip of sheath 74, a pusher 82, and a housing 72. The sheath 74 may be advanced entirely through the tubular body 29, with the needle extending out of the launch tube to pierce through tissue and deploy an anchor. The pusher 82, which may be a flexible wire or hypotube, is slidably positioned within the sheath 74 and is connected to an actuator 80 in the housing 72. Pushing or turning the actuator 80 advances or retracts the pusher 82 relative to the sheath 74 for deploying the anchors from an opening 78 in the needle 76.

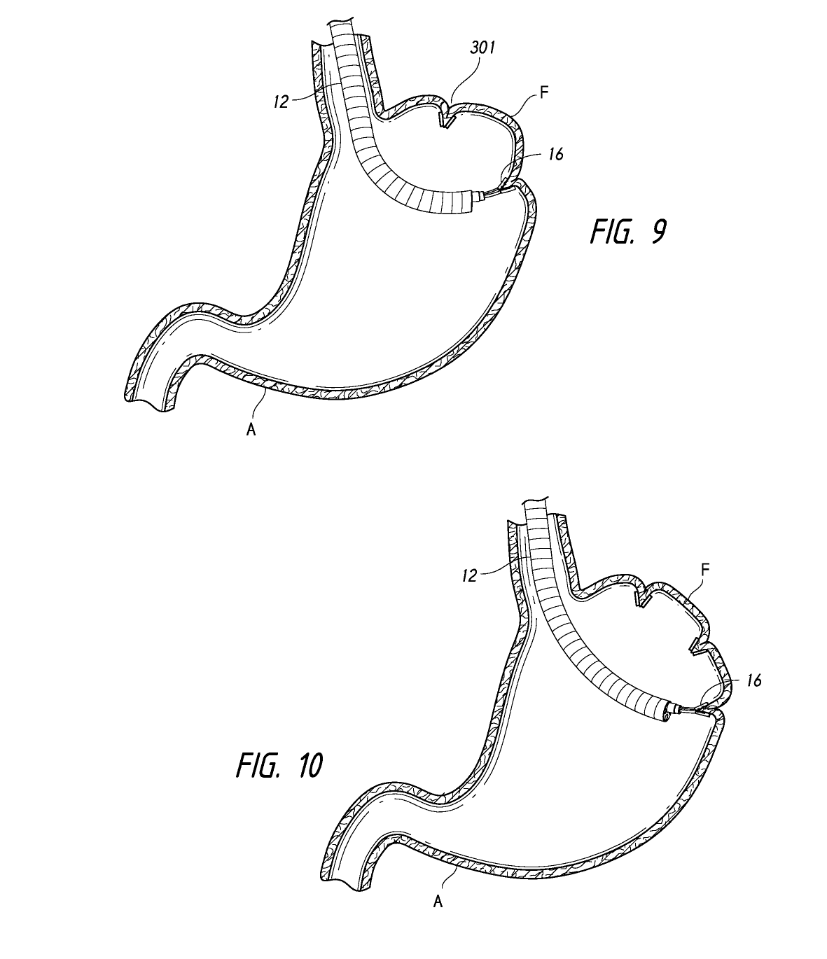

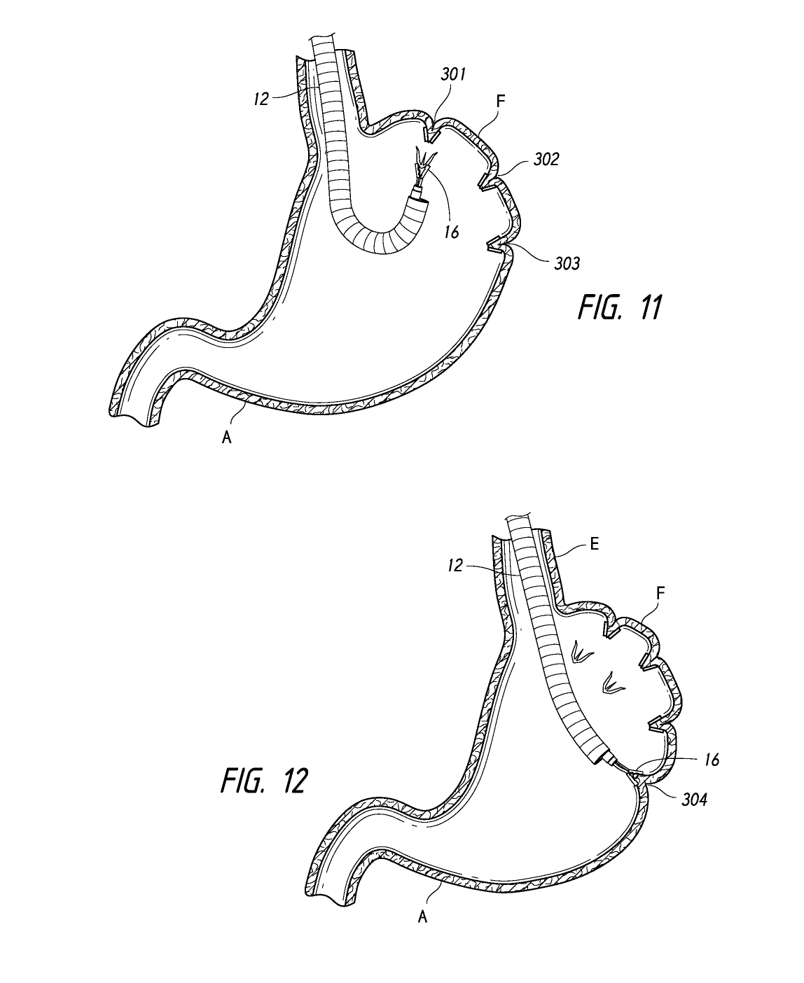

As shown in FIGS. 2 and 3, a tissue anchor assembly 100 is positioned in front of the pusher 82 within the sheath 74 for deployment from the sheath 74. The tissue anchor assembly 100 may include a pair of tissue anchors 50a, 50b slidably retained by a connecting member, such as a suture 60, as shown in FIG. 13. A locking mechanism, such as a cinch 102, is also slidably positioned on the suture 60. The cinch 102 is configured to provide a cinching force against the proximal anchor to hold tension on the suture. Accordingly, the tissue anchor assembly 100 is adapted to substantially permanently hold a fold of tissue, as shown in FIG. 14.

Referring still to FIGS. 2 and 3, after the anchor assembly 100 has been fully deployed from the sheath 74, the spent needle deployment assembly 70 may be removed from the tissue manipulation assembly 27 and another needle deployment assembly may be introduced without having to remove the tissue manipulation assembly 27 from the patient.

As shown in FIGS. 4, 5 and 6, the tissue manipulation assembly 27 is located at the distal end of the tubular body 29 and is generally used to contact and form tissue folds or plications. The tissue manipulation assembly 27 may be slid through a lumen in the endoscopic system 12 while in a low-profile configuration, e.g., trans-orally, trans-anally, percutaneously, through an endoscope, an endoscopic device, directly, etc. The surgical site 36 as well as the procedure may be viewed through the visualization lumen 30 or a separate imager.

FIGS. 4-6 and 13-14 illustrate use of the endoscopic system 12 to form plications. As shown in FIG. 4, the tissue manipulation assembly 27 and the tissue engagement member 32 are advanced distally out from the endoscopic system 12 through lumens 26 and 28. The tissue engagement member 32 is advanced into contact against the tissue, and is then rotated until the tissue is engaged. The engaged tissue F is pulled back or proximally relative to the endoscopic system 12. Referring to FIG. 5, the launch tube 40 is pulled back to open the jaws. This rearward movement of the launch tube 40 may also urge the lower jaw 18 to pivot to an angle relative to a longitudinal axis of the tubular body 29. The launch tube may optionally be temporarily locked into open position, keeping the jaws locked open. The open jaws are manipulated around the engaged tissue. Referring to FIG. 6, the launch tube 40 is pushed forward or distally, to close the jaws. The lower jaw may be maintained at an angle relative to the tissue to further facilitate manipulation of the grasped tissue. The jaws may also optionally be temporarily locked in the closed position.

Referring to FIGS. 3, 13 and 14, an anchor deployment assembly 70 is moved through the handle with the needle 76 at the distal end of the anchor deployment assembly extending out through the launch tube 40. The needle 76 may pass through an opening in the upper jaw to pierce through the grasped tissue. A first tissue anchor 150A is then deployed on the distal side of the engaged tissue. The needle is withdrawn out from the tissue and a second tissue anchor 150B is deployed on the proximate side of the engaged tissue. The suture is tensioned and a cinch 102 is pushed up against the proximal tissue anchor 150B, forming a substantially permanent tissue fold or plication, as shown in FIG. 14.

Referring back to FIG. 6, the tissue engagement member 32 may be retracted from the tissue F or it may be left within the tissue while the tissue manipulation assembly engages and secures the tissue F. The tissue engagement member 32 is shown as a tissue piercing helix or corkscrew structure on a flexible shaft 34, although other tools for engaging tissue, including jaw tools, may also be used.

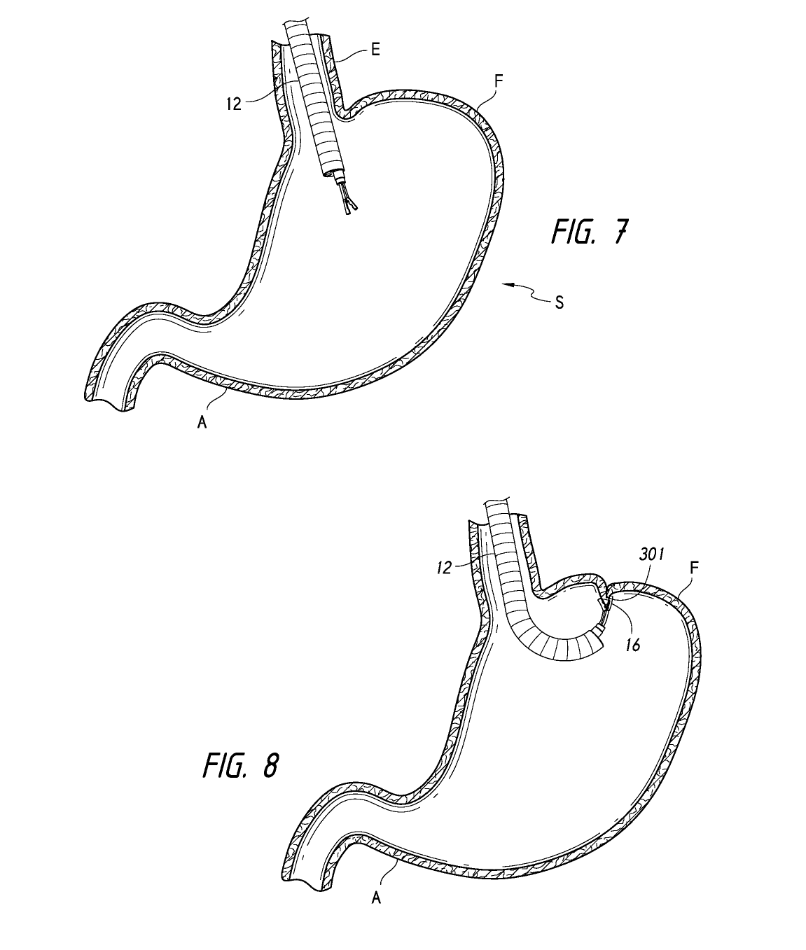

FIGS. 7-12 show formation of multiple plications in stomach using the steps described above. In FIG. 7, the endoscopic system 12 is advanced per-orally, through the esophagus E, and into the stomach. Generally, the steps described here are performed using a scope, such as the scope 30 in the endoscopic system 12. The scope allows the surgeon to see the surgical site, as well as the tools acting on it. As shown in FIG. 8, a first tissue fold or plication 301 is formed. FIGS. 9-12 show movement of the endoscopic system 12 within the stomach to form additional plications 302-304. The plications are spaced apart typically by about 1-5 or 2-4 cm to allow for subsequent drawing the plications together to form a contracted tissue area, as described below.

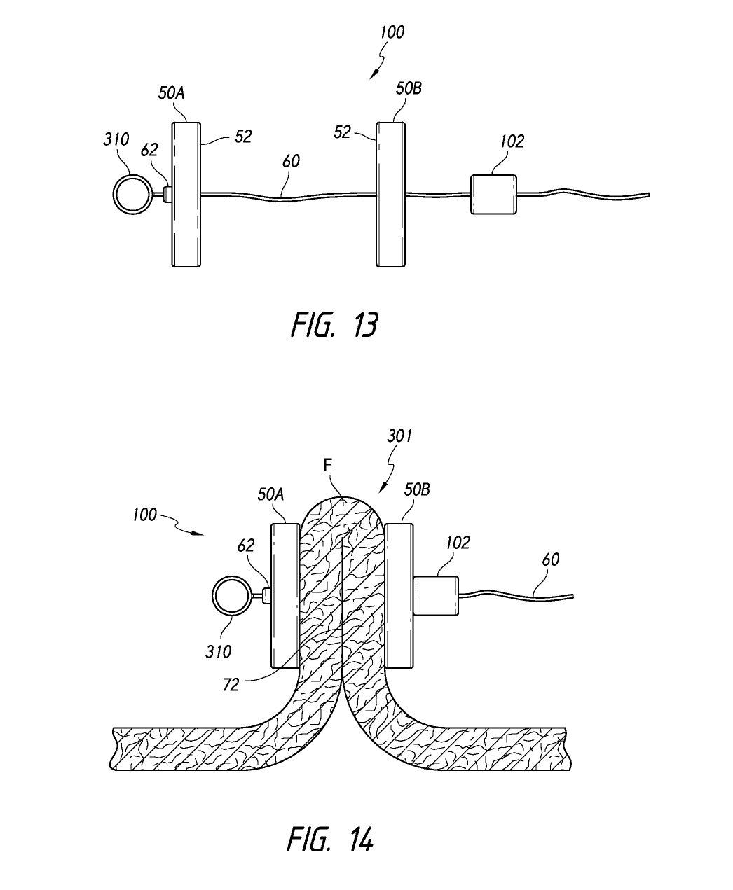

FIG. 15 shows the stomach of a patient after eight plications 301-308 have been formed. Formation of the plications may be described as a first phase of surgical method. A loop or ring 310 is provided at each plication. The loop 310 may be attached to, or made as part of, either one of the anchors 150 used to form the plications as shown in FIGS. 13-14. The loop 310 may alternatively be attached to, or made part of, the suture 60 connecting the anchors 150. The loop 310 may be suture material, or a plastic or metal component of the anchor assembly 100. The plications 301-308 are allowed to heal over a healing interval of one, two, four, or eight weeks, or longer. During this interval, the tissue heals and generally forms scar tissue at each plication, creating localized strengthened areas at the plications. In some cases, the healing step may be largely omitted and the plications may be formed and tensioned in a single operation.

FIG. 16 shows the second phase of the method. In this second phase, a suture or wire, referred to here as a loop suture 312, is threaded through the loop 310 of two or more of the plications and then tensioned and tied off. This pulls the plications together reducing the size and shape of the stomach. Since the tissue at the plications may already be healed during this phase of the method, the tissue is in better condition to withstand the pulling forces exerted. Alternatively, the looping step may be performed at the same time as the first step. In addition, moving the two or more plications closer together, or into contact with each, allows for a greater reduction in the size of the stomach, in comparison to forming plications alone. In the example shown, the loop suture 312 is threaded sequentially through the loops 310 of the anchor assemblies at plications 302, 303, 306, 307 and 301.

The number of plications used, the positions of the plications, the number of loop sutures used, and the sequence or pattern of plication connections made by a loop suture, may of course vary by patient, condition, and the surgeons preference. In the example shown in FIG. 17, loop suture 312A is routed through the loops 310 at plications 301, 302, 303 and 304, in that order. Similarly, loop suture 312B is routed through the loops 310 at plications 305, 306, 307 and 308, in that order, and in this case, independent of loop suture 312A. In FIG. 17, a first or leading end of the loop suture 312A is routed through the loops at the desired plications, and is then brought back through the endoscopic system 12 and out of the patient's body.

A loop or eyelet 314 is then tied or otherwise formed or attached to the leading end of the loop suture 312A. The second or back end of the loop suture 312A is threaded through the eyelet 314. The surgeon then pulls on the second end of the loop suture 312A, which pulls the eyelet 314 back down through the endoscopic system 12, and into the stomach. FIG. 17 shows the eyelet 314 in the stomach near plication 301. Further pulling on the loop suture 312A draws plications 301-304 towards each other. With the loop suture 312A appropriately tightened, a cinch or knot is pushed forward, using a knot pusher or similar tool, towards or up against plication 301 to prevent the loop suture 312A from loosening. The length of loop suture behind or proximal to the cinch or knot is then cut and removed.

In the examples above, the loop suture 312 is routed through the loops 310 in the second phase of the method. However, this step may alternatively be performed in the first phase of the method, as part of the same surgical procedure used to form the plications. In this case, the loop suture 312 is left loose in the stomach during the healing interval, to avoid stressing the plications. The second phase of the method is then simplified as the loop suture 312 is already in place and only needs to be tensioned and tied off.

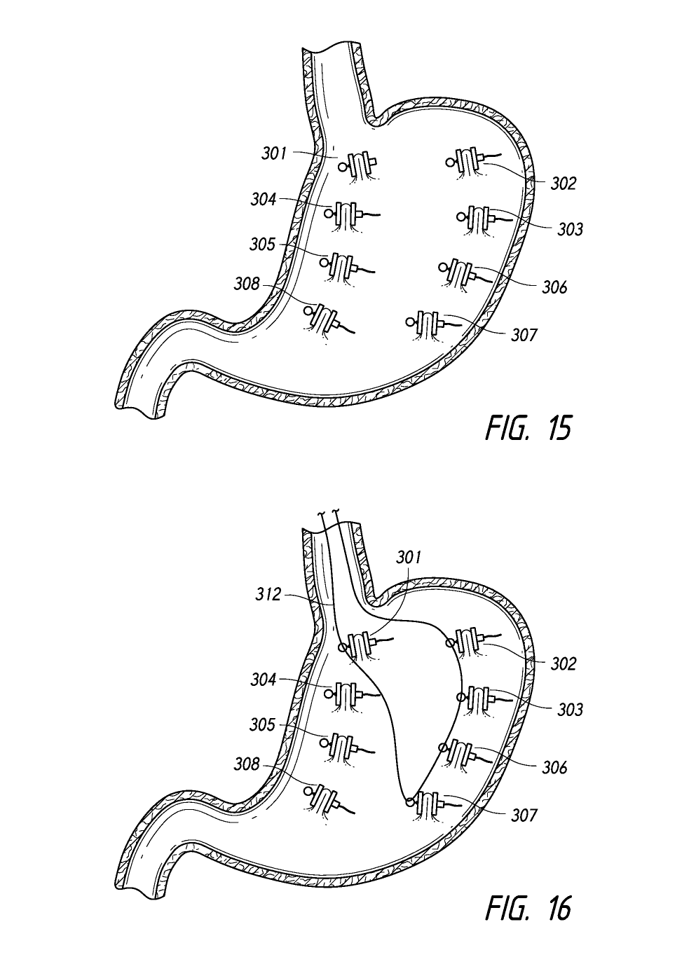

FIGS. 18-20 diagrammatically show the movement of the plications. FIG. 18 shows plications 301 and 302 after the healing period. The loop 310 of plication 301 is facing the loop 310 of plication 302. Typically, the loops 310 will be positioned this way when the plications are formed. Precise alignment of the loops is not necessary as the plications have a certain amount of flexibility, and the precise angle between the loop suture 312 connecting them is not critical. The forces exerted on the plications can be better managed however if the misalignment of the loops 310 is under about 90, 45 or 30 degrees.

FIG. 19 shows the plications 301 and 302 with the loop suture 312 threaded through the loops 310, before tensioning, and with the plications still in their original positions. FIG. 20 shows the plications after tensioning the loop suture 312. The plications are now drawn towards each other, with a knot or cinch 316 pushed into place to hold the plications next to each other, forming a contracted tissue area generally indicated at 318. The contracted tissue area 318 is formed by the plications moved towards each other via the anchor assemblies 100 and the loop suture 312 and cinch 316. Most of the volume in the open space between the plications in FIG. 19 is closed up with the formation of the contracted tissue area 318 in FIG. 20. The volume of the stomach can be significantly reduced by first forming plications and then forming contracted tissue areas. FIG. 20 shows a contracted tissue area 318 having two plications. However, contracted tissue areas may also be formed using greater numbers of plications pulled towards each other via a loop suture 312.

In similar alternative methods, it is not necessary for both ends, or even one end, of the loop suture 312 to run back out of the body via the endoscopic system 12 to provide an eyelet or cinch for holding the plications into contracted positions. As shown in FIG. 21 for example, the loop suture 312 may be replaced by a plastic or metal locking ring, ratcheting ring, or zip tie component 320. Using gripping and/or other end effectors of the endoscopic system 12, the lead end 322 of the component 320 is threaded through the loops 310. The lead end is then inserted through the lock end 324 of the component, to hold the plications together. As used here, the term together means that the plications have been moved towards each other from their original positions, with or without actually touching each other.

Turning to FIG. 22, a knot 328 and a first anchor 330 are provided on the back end of a modified loop suture 326. The leading end of the loop suture 326 is guided through the loops 310 of the anchor assemblies 100 of plications 301 and 302. A second anchor 332 and a cinch 334 are then deployed onto the leading end of the loop suture 326. The loop suture 326 is tensioned, pulling the plications together. The second anchor 332 and the cinch 334 are then pushed down the loop suture 326, to hold the plications together, as shown in FIG. 23.

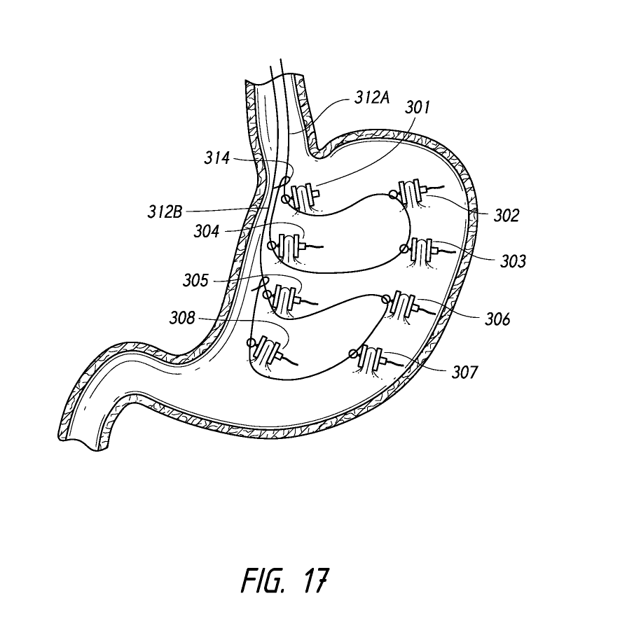

FIG. 24 shows modified loop suture assembly 340 having expandable or umbrella anchors 342 which are held in a folded or collapsed position, for example within a delivery catheter 348 which is guided through the loops 310. A leading end of a length of suture or wire 344 of the loop suture assembly 340 has a knot 328 or similar obstacle on the suture which prevents forward or distal movement of a first umbrella anchor 342 along the suture. A second umbrella anchor 346 is positioned on the suture 344 behind or proximal to the first umbrella anchor 342.

In use, the leading end of a catheter or other delivery device is passed through the loops 310. The first anchor 342 is then deployed, or pushed out of the delivery device 348 and opens, unfolds, or expands to a size greater than the diameter or other characteristic dimension of the distal loop 310A shown in FIG. 24. The delivery device or tube 348 is then pulled back through both loops 310 and the second anchor 346 is deployed. The second anchor then similarly opens to a size preventing it from passing back through the proximal loop 310B. The suture 344 is tensioned, pulling the plications together, and a cinch 316 is advanced up against the second anchor 346, forming a contracted tissue area 318.

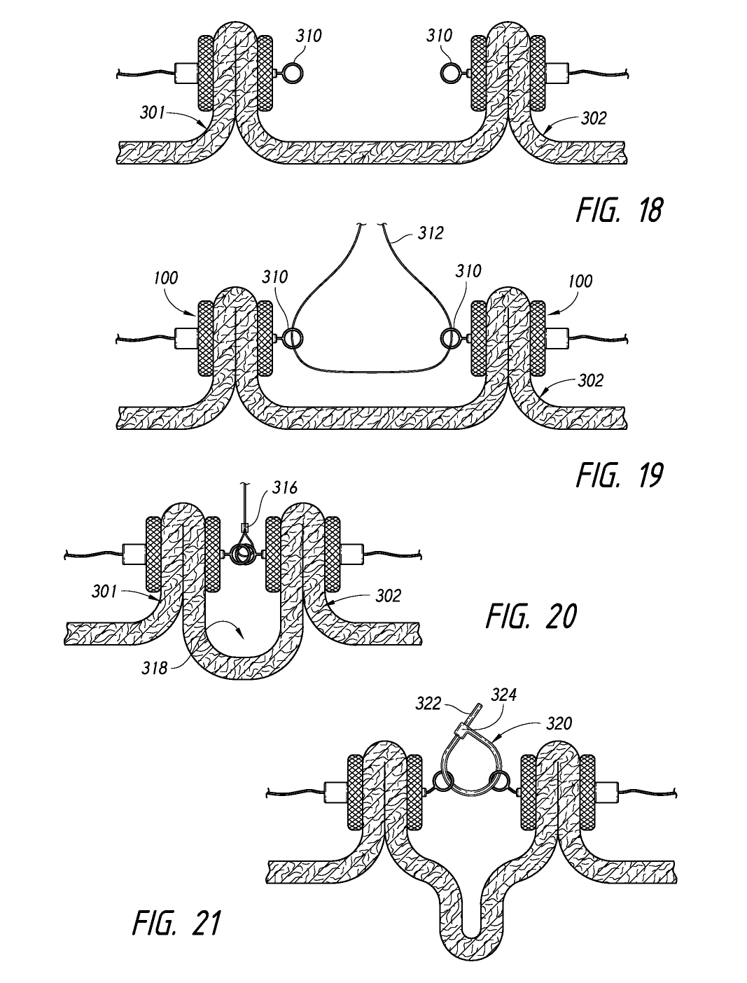

FIGS. 25-29 show an alternative method where an end loop 352 of a threading suture 350 is passed over the loop 310 at the plication 302. The proximal end 354 of the threading suture 350 is then threaded through the loop 310, as shown in FIG. 27. The end loop 352 and the loop 310 on the anchor at the plication are then interlocked, as shown in FIG. 28. As shown in FIG. 29, the threading suture 350 is tensioned and a cinch 316 larger than the diameter of the proximal loop 310 is advanced along the threading suture 350 to form a contracted tissue area 318. Alternatively, an anchor 330, shown in dotted lines in FIG. 29, may be placed in front of a smaller cinch, with the anchor preventing the cinch from passing through the loop 310.

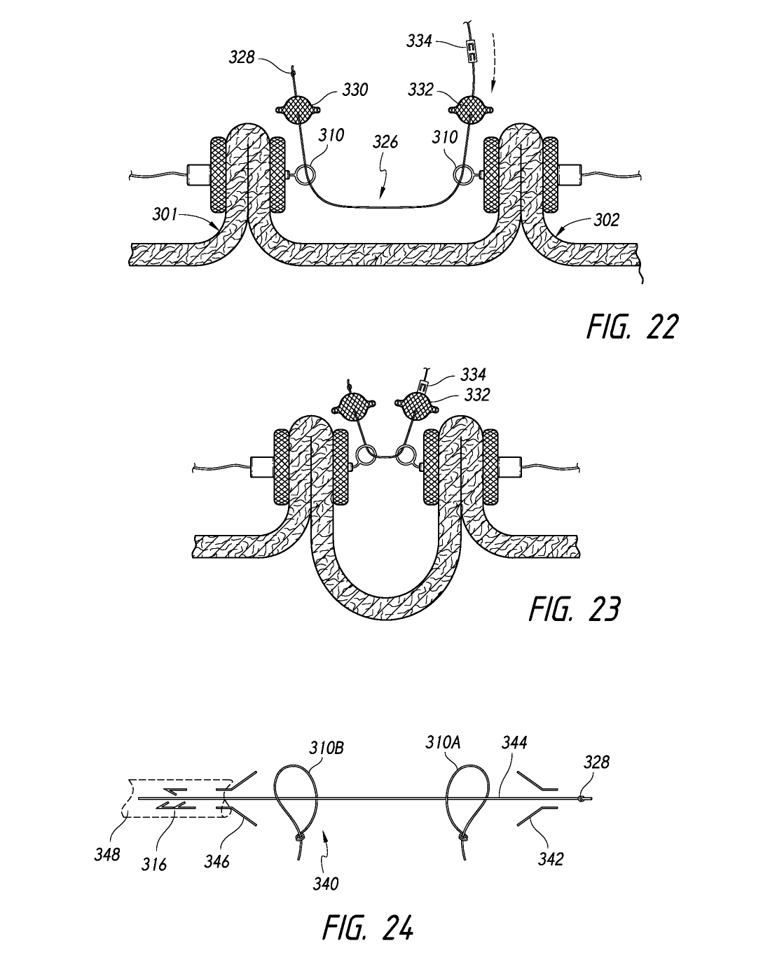

Turning to FIGS. 30 and 31, an anchor assembly 354 may have two or more pairs of anchors 330, 332 attached to suture 60. Multiple anchor assemblies 354 may also be combined to form contracted tissue areas 318 using the suture 60 of the anchor assemblies. In FIG. 30, a loop 310 is formed by tying a loop knot 360 in the suture 60. The loop knot 360 forms the suture into a loop 310, and also provides a mechanical stop for the distal anchor 332 during the cinching step. To more securely maintain the loop knot 360, cyanoacrylate adhesive may be applied to the knot. Typically, the loop 310 has a diameter of about 0.1 to 0.8 or 0.2 to 0.5 inches.

The loop 310 may be placed anywhere along the suture 60 in the anchor assembly. FIG. 11 shows an anchor assembly with a loop 310 formed by making a loop knot 360 at the proximal end of the suture 60, and with a conventional stop knot 362 at the distal end.

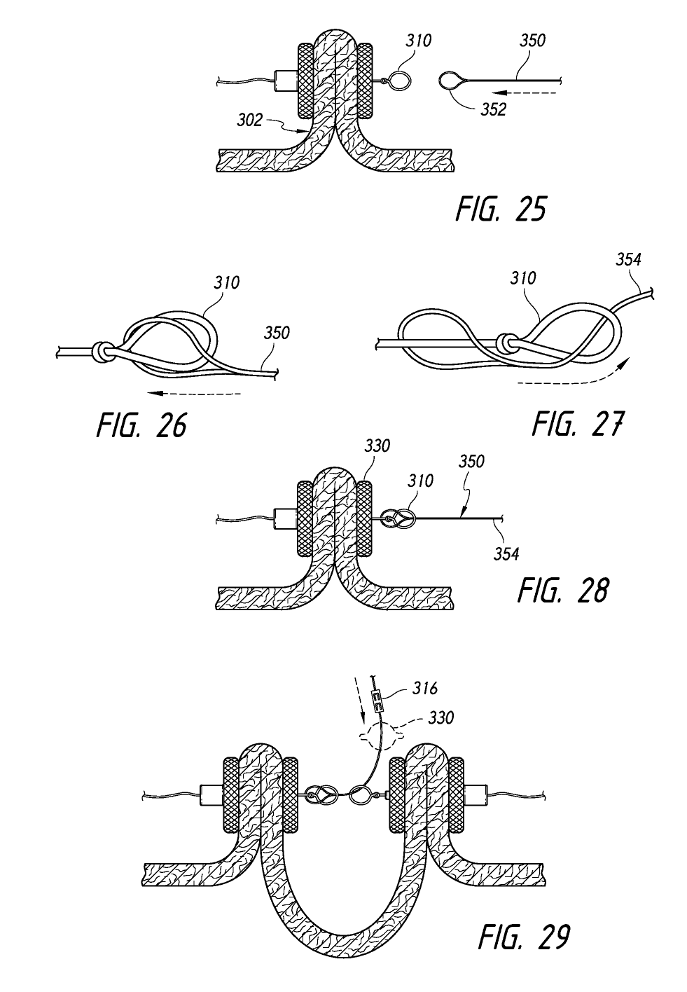

As shown in FIGS. 32-34, a cinch 370 may have a cinch ring 372 to provide a loop on the anchor assembly. Alternatively, the loop 310 may be provided by another separate component on the anchor assembly, such as flexible metal ring, e.g., a nitinol ring, attached to the distal end of the anchor assembly. The flexible metal ring may remain folded or compressed when loaded inside of a delivery catheter, and then expand when deployed.

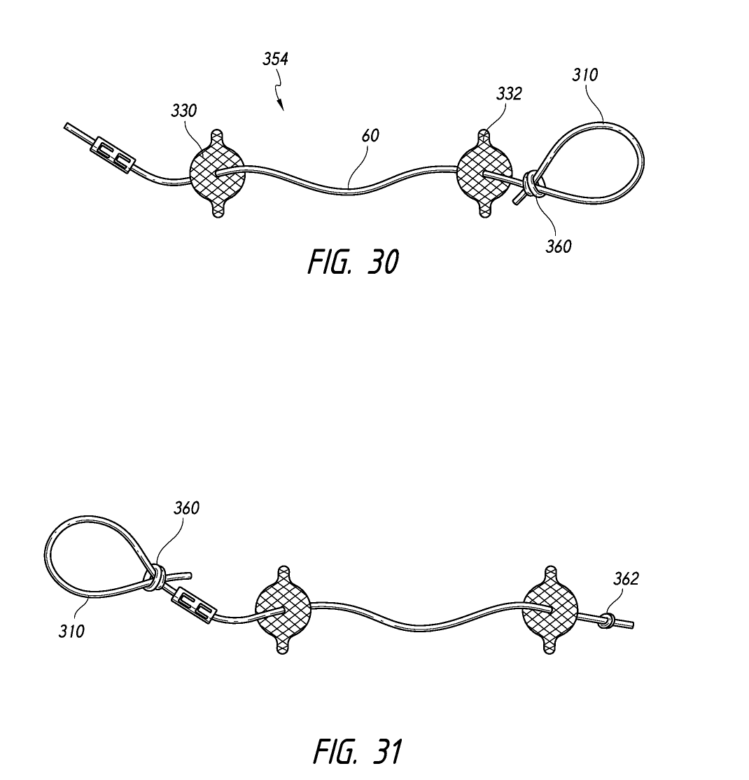

FIG. 35 shows a design where the leading end of the loop suture 312 is attached to a stiffener or end piece 380. The stiffener allows the leading end of the loop suture to be more easily grasped, held, and passed through the loops 310.

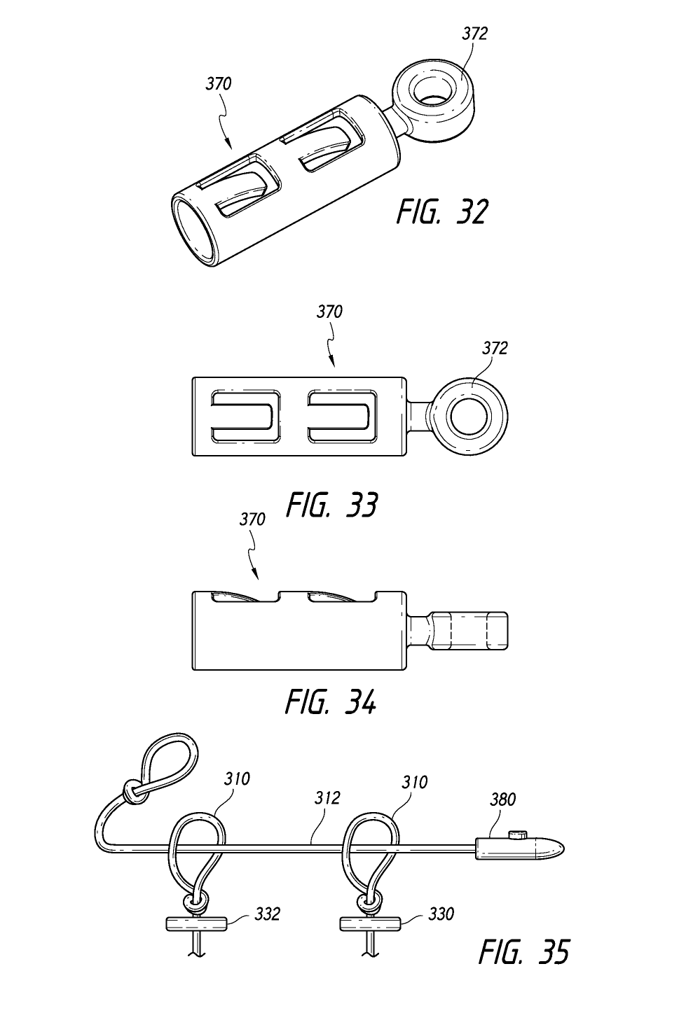

FIGS. 36-39 show a threading or routing tool 390 and a method for threading the loop suture 312 through the loops 310 of the anchor assemblies 100, 340 or 354. The threading tool 390 may be advanced into the stomach, or other surgical site, via the endoscopic system 12 shown in FIG. 1. A grasper 402 runs through a lumen 394 in the tool 390. Jaws or a similar grasping device 406 are provided at the distal end of the grasper 402, with the jaws operated using a controller 218 or other device outside of the patient's body. A loop slot 398 is provided in a cylindrical end 392 of the tool 390. A receiver hole 396 is aligned with the lumen 394 on an opposite side of the loop slot 398. A scope 400 may be included in the tool 390, to assist in visually determining the position of the loop 310 during threading of the loop suture 312.

In use, as shown in FIG. 36, a loop suture 312 having an end stiffener or end piece 380 is preloaded into the tool 390, with an end ball 404 or other easily grasped and released element on the end piece 380. The end piece 404 is held by the jaws 406. The loop 310 of a plication is positioned into the loop slot 398, by moving the cylinder end 392 of the tool 390, and/or the loop 310. The loop 310 may optionally be moved or positioned into the loop slot 398 using an assisting tool separate from the threading tool 390, and also optionally separate the endoscopic system 12. Typically though, any assisting tool used will be advanced to the surgical site within the endoscopic system 12.

Moving to FIG. 37, in the next step, the grasper 402 is advanced distally into the receiver hole 396, to move the end piece 380 through the loop 310. As shown in FIG. 38, the grasper 402 then releases the loop suture 312 and is pulled back through the loop, with the loop suture 312 remaining in the receiver hole 396. The tool 390 and/or the loop 310 are then moved away from each other, with the loop suture 312 now threaded through the loop, as shown in FIG. 39. These steps are then repeated on subsequent loops, until the loop suture 312 is threaded through all of the loops designated for forming contracted tissue area. The grasper 402 is then pulled back out of the lumen 394, while grasping the end piece 380 or similar component attached to the leading end of the loop suture 312. The endoscopic system 12, or other catheter used to move the tool 390 to the surgical site, is then removed from the patient's body, so that the both ends of the loop suture 312 can be brought together, as described above. If the loop suture does not have any end piece 380, the jaws 406 or other holding device may grasp directly onto the leading end of the loop suture 312, and perform the same steps described above.

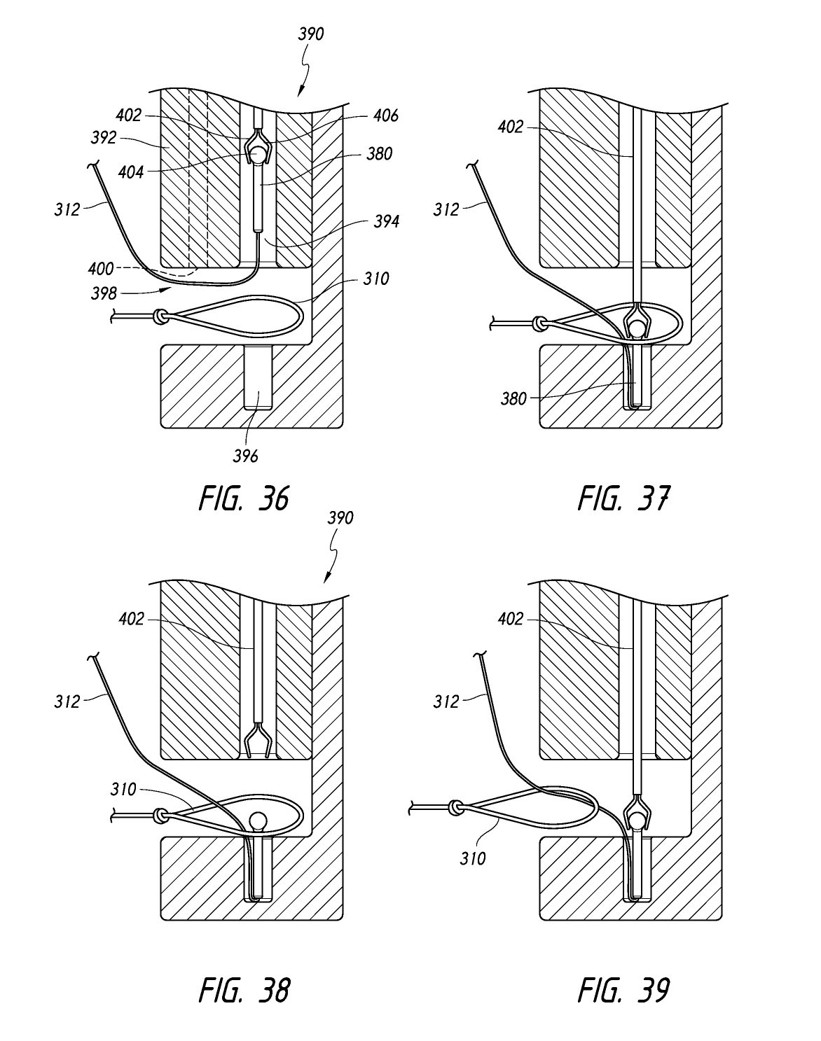

FIGS. 40-43 show another threading tool 410 having a hook 412 pivotally attached to an extension 416. In this design, the hook 412 is initially in the open position shown in FIG. 40. The hook 412 is moved through a loop 310, as shown in FIG. 41. The hook 412 is then pivoted into a closed position shown in FIG. 41, via operation of a link or wire 414. The grasper 402 then advances, or moves down through the lumen and places the leading end of the suture loop 312, or an end piece 380 attached to the suture loop 312, into a receiving hole in the hook. The hook 412 is then moved back into the open position as shown in FIG. 42, threading the leading end of the loop suture 312 through the loop 310. The grasper 402 is withdrawn. The leading end of the loop suture 312, or any end piece 380 attached to it, remain on the hook 412. The hook 412 is returned to the closed position, as shown in FIG. 43. The steps described above are repeated for the next loop 310 at the next plication. When the leading end of the loop suture 312 has been threaded through all selected loops, the grasper 402 is advanced and grasps the end piece 380, which can then be pulled back out of the tool 410, to bring the ends of the loop suture together.

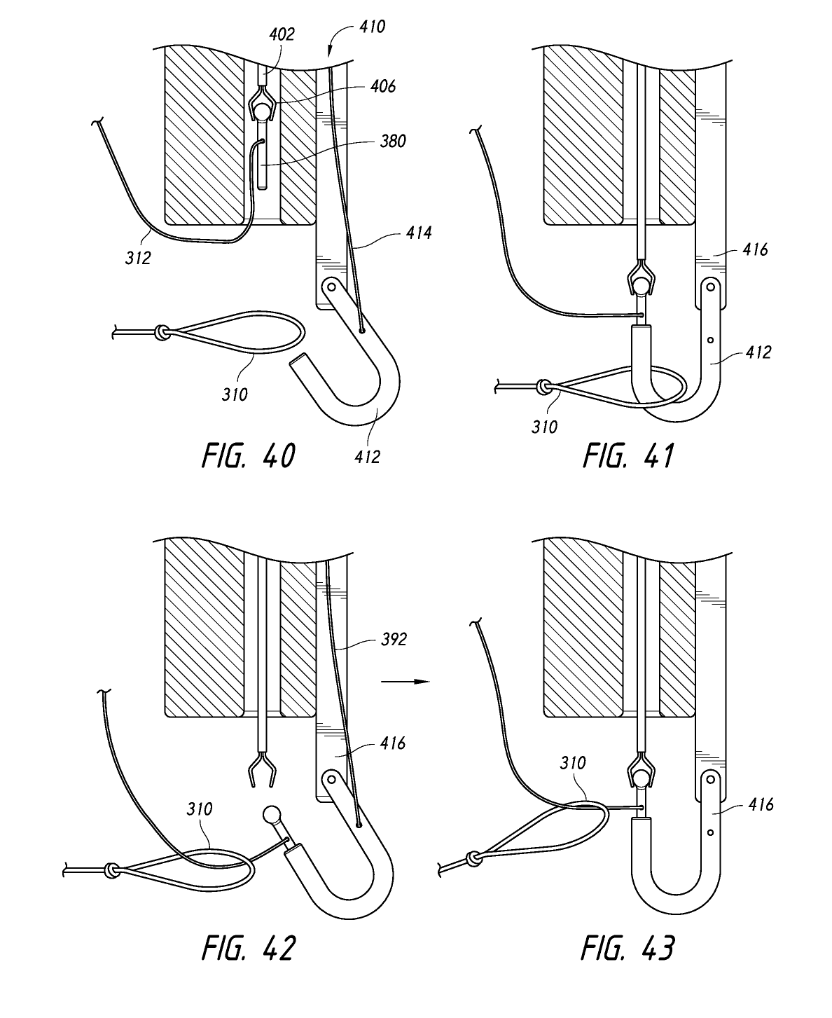

FIGS. 44-46 illustrate another tool 420 which may be used to thread a loop suture 312 through one or more loops 310. The tool 420 has a first grasper 422 and a second grasper 424, with both graspers on arms 426 pivotally attached to the tool body. The graspers may be spring biased into an open position and selectively closed by pulling on a control wire. In FIG. 44, the arms 426 are open. The first grasper 422 grasps an end piece 380 on the loop suture 312. The arms are then closed, bringing the end piece 380 through the loop 310, and into contact with the second grasper 424. The second grasper 424 grasps the end piece 380 and the first grasper releases the end piece, as shown in FIG. 45. The arms are then opened with the second grasper 424 holding the end piece 380, and threading the loop suture 312 through the loop. The graspers 422 and 424 may optionally be made part of the arms 426.

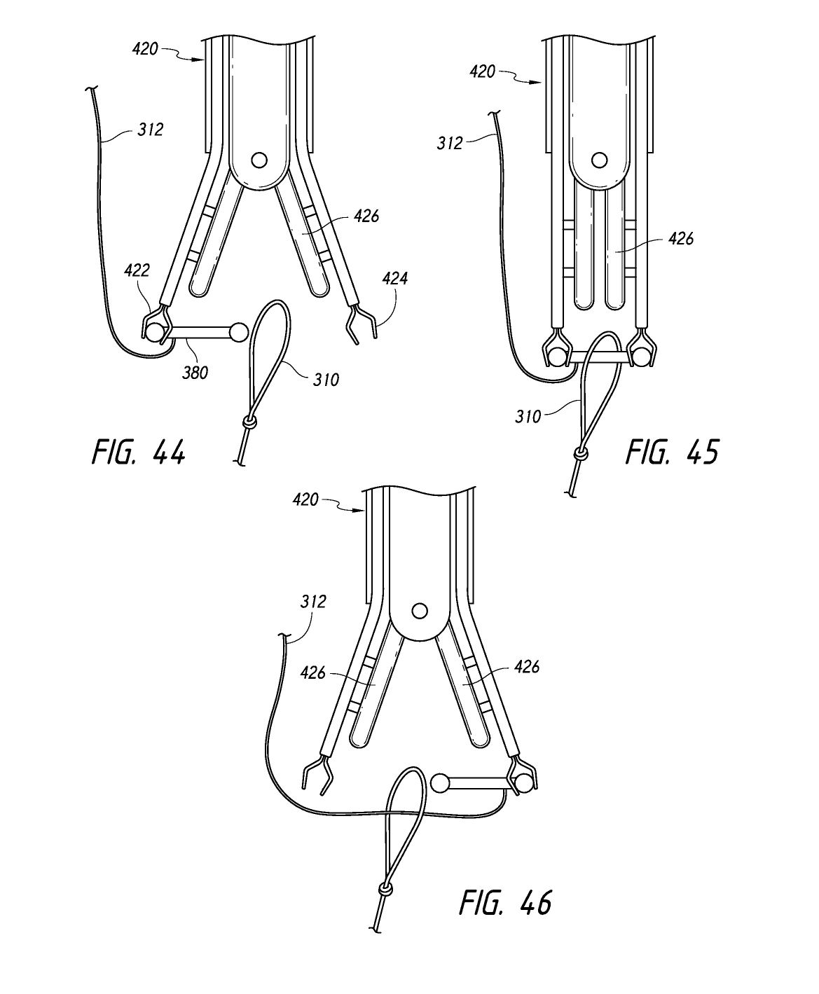

FIGS. 47-49 show an alternative tool 430 that also has a pair of arms pivotally attached to a tool body, as with the tool 420 described above. However, the tool 430 has openings 432 and 434 in each arm adapted to receive a suture bar 440 on or near the end of a loop suture 312. In use, the tool 430 is deployed with the suture bar 440 partially or fully within the second opening 434 in the second arm. A second retainer 438 secures the suture bar 440 in the opening. The second retainer 436 may be a wire slidably positioned with the arm, and movable between an advanced position, shown in FIG. 47, where the retainer is engaged with, and holds the suture bar 440, and a retracted position, shown in FIGS. 48 and 49, where the retainer 438 is withdrawn, allowing the suture bar 440 to move out of the opening 434. The suture bar 440 may have a dumbbell shape, a shown in FIG. 47, or it may be generally cylindrical in the same way as the end piece 380 shown in FIG. 15, or it may have a bulge or ball one on one end only. The suture bar 440 and the openings 432 and 434 may optionally be curved, to better match the arc of the arm movement.

Referring to FIG. 47, the open arms are positioned on opposite sides of a loop 310. The arms are then closed, as shown in FIG. 48. The suture bar 440 is then positioned within both openings 432 and 434. The second retainer 438 is pulled back or released, and the first retainer 426 is advanced or engaged on to the suture bar. The arms are then opened, with the suture bar passed from the first arm, through the loop, to the second arm. The retainers may be automatically controlled via a mechanism in the handle 31 that operates the retainers with movement of the arms.

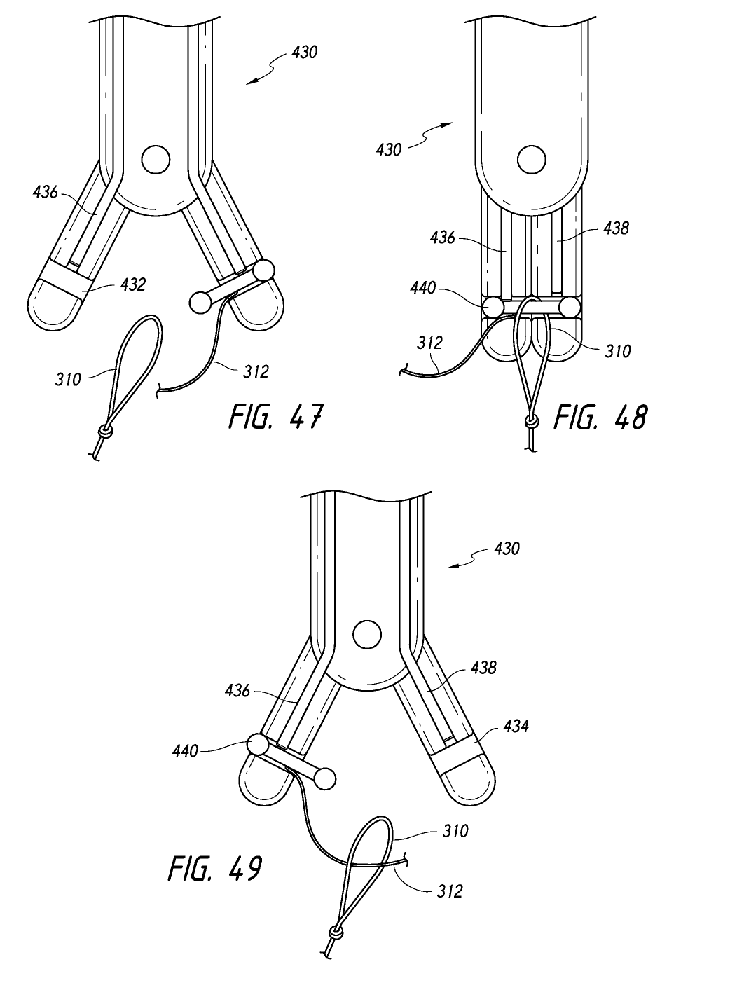

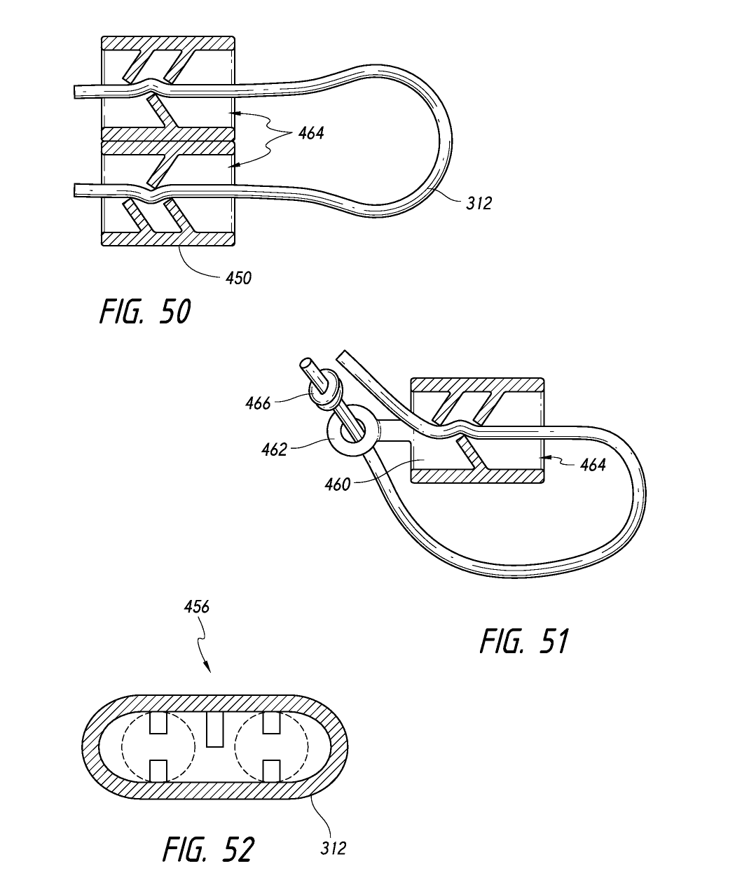

The ends of the loop suture 312 may be joined by a sliding knot that can be pushed towards or in between the plications to form a contracted tissue area 318. Alternatively, the ends of the loop suture 312 may be routed through a cinch. FIG. 50 shows a cinch 450 for tightening the ends of the loop suture 312. The cinch 450 has two passageways 464, one for each end of the loop suture 312. Both passageways have internal vanes, arms, or similar devices which allow the suture to move only in one direction through the passageways, i.e., in the tightening direction. FIG. 52 shows another cinch 456 having an oval or flattened cross section, but that may otherwise be similar to the cinch 450 shown in FIG. 50. FIG. 51 shows a cinch 460 having an eyelet 462. In this design, the leading end of the loop suture 312 is passed through the eyelet 462, and then through the passageway 464. A sliding knot 466 is positioned distally of the eyelet.

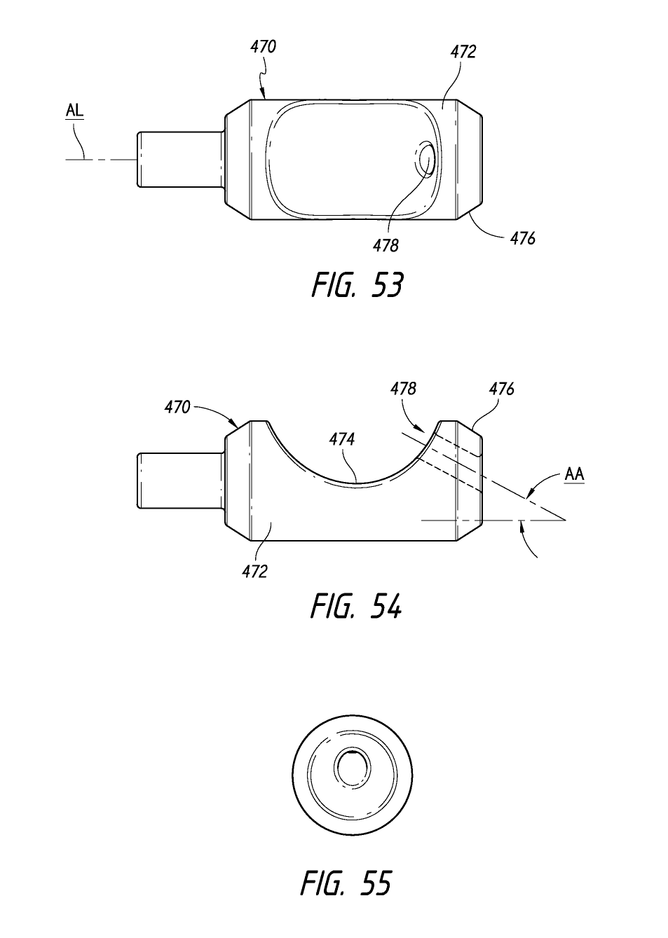

FIGS. 53-55 show an endoscopic knot pusher 470 which may be used to push a knot on the loop suture 312. The knot pusher 470 has a generally cylindrical head 472 with radius or curvature 474 cut into one side, and with the cylindrical head 472 having a beveled front end 476. A through hole 478 extends from the radius 474 through and out of the front end of the head 472. The hole 478 is oriented at an acute angle AA to the longitudinal axis AL of the head, with the angle AA ranging from about 10 to 30 degrees. The hole diameter for use with commonly used suture is about 0.02 to 0.06 or 0.03 to about 0.05 inches.

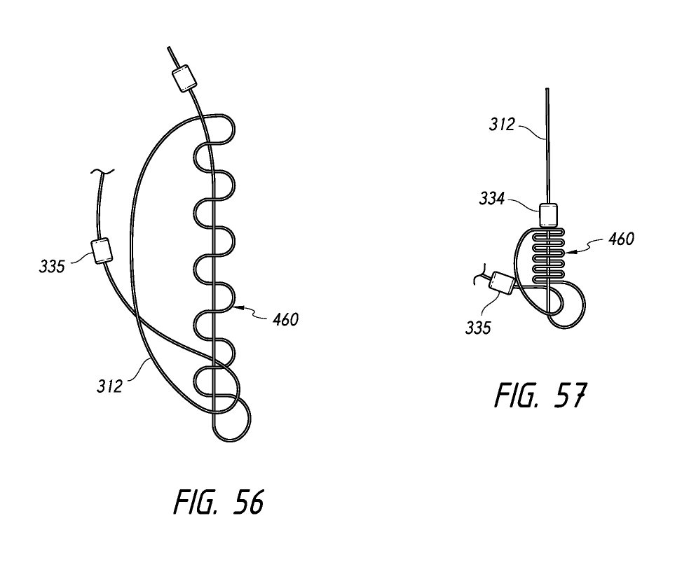

FIGS. 56-57 a design for backing up a slip knot 460 with a cinch 334, which may be used in the methods described above. The cinch 334 may be used as back up for the slip knot 460, to increase resistance to breaking or backing off. A second cinch 335 may optionally be provided on the leading end of the loop suture 312 (in front of the knot 460) to similarly increase break strength and prevent the knot from backing off during cycling.

Thus, novel devices and methods have been shown and described. Various changes and substitutions may of course be made without departing from the spirit and scope of the invention. The invention, therefore, should not be limited except by the following claims, and their equivalents.

1. A surgical method, comprising:
forming a first plication in tissue of a patient using a first tissue anchor assembly having a first loop;
forming at least a second plication in the tissue of the patient using a second tissue anchor assembly having a second loop;
allowing the plications to heal;
moving the first and at least a second plications relatively towards each other; and
passing a holding element through the first and at least the second loops to hold the plications together.

2. The method of claim 1 further comprising moving the first and at least a second plications towards each other by pulling the first loop relatively towards the second loop.

3. The method of claim 1 comprising moving the first and at least a second plication relatively towards each other by passing a loop suture through the first loop and through the second loop, and tensioning the loop suture.

4. The method of claim 1 wherein the holding element comprises a loop suture passed through the first and at least a second loop.

5. The method of claim 1 comprising allowing the plications to heal for at least two weeks.

6. The method of claim 1 with the holding element comprising a zip tie.

7. The method claim 1 with the holding element comprising wire.

8. The surgical method of claim 4 further comprising bringing first and second ends of the loop suture out of the patient, providing a knot or a cinch on the first and second ends of the loop suture, and pushing the knot or cinch along the suture to a position adjacent to at least one of the first and second plications.

9. The surgical method of claim 4 further comprising threading a distal end of the loop suture through a loop at the proximal end of the loop suture, and bringing the leading end out of the patient.

10. The surgical method of claim 4 further comprising providing a first tissue anchor on a proximal end of the loop suture, and with the first tissue anchor held against the first anchor.

11. The surgical method of claim 4 with the first anchor comprising an umbrella anchor.

12. The surgical method of claim 4 further comprising securing the loop suture by advancing a cinch and a tissue anchor against the first or second loop, with the tissue anchor preventing the cinch from passing through the first or second loop.

13. The surgical method of claim 4 further comprising securing the loop suture by advancing a cinch against the first or second loop, with the cinch having a characteristic dimension that prevents it from passing through the first second loop.

14. The surgical method of claim 14 with the cinch further comprising a cinch ring, and further comprising threading the distal end of the loop suture through the cinch ring.

15. The surgical method of claim 4 moving a loop suture through the first loop and through the second loop, before allowing the plications to heal.

16. The surgical method of claim 1 further comprising:
forming a third plication in tissue of the patient using a third s, e anchor assembly having a third loop;
moving the loop suture through the third loop; and
tensioning the loop suture to displace at least two of the first, second and third plications.

17. The surgical method of claim 4 with the distal end of the loop suture having a rigid end piece, and further comprising grasping the rigid end piece and passing it through the first and second loops.

18. A surgical method, comprising:
forming a first plication in tissue of a patient using a first tissue anchor assembly having a first loop;
forming at least a second plication in the patient using a second tissue anchor assembly having a second loop;
passing a holding element through the first and at least a second loops and temporarily attaching a first end of the holding element to a second end of the holding element or to at least one of the loops; and, at a later time,
pulling on the holding element to move the first and at least second plications relatively towards each other; and
knotting or cinching the holding element to hold the plications together.

19. The method of claim 18 with the holding element comprising a loop suture.

20. A method for treating obesity, comprising:
advancing a delivery catheter through a patients mouth and esophagus and into the patient's stomach;
forming a first plication in the tissue of the stomach using a first tissue anchor assembly having a first loop, and first and second tissue anchors connected by a first suture, by:
forming a first tissue fold;
passing a needle through the first tissue fold;
deploying the first tissue anchor from the needle on a distal side of the first tissue fold;
withdrawing the needle back through the first tissue fold;
deploying the second tissue anchor from the needle on a proximal side of the first tissue fold;
tensioning the first suture to pull the first tissue anchor towards the second tissue anchor;
securing the second tissue anchor onto the first suture to prevent the first tissue anchor from pulling away from the second tissue anchor; and
forming a second plication in the tissue of the stomach using a second tissue anchor assembly having a second loop, and third and fourth tissue anchors connected by a second suture, by
forming a second tissue fold;
passing a needle through the second tissue fold;
deploying the third tissue anchor from the needle on a distal side of the second tissue fold;
withdrawing the needle back through the second tissue fold;
deploying the fourth tissue anchor from the needle on a proximal side of the second tissue fold;
tensioning the second suture to pull the third tissue anchor towards the fourth tissue anchor;
securing the fourth tissue anchor onto the second suture to prevent the third tissue anchor from pulling away from the fourth tissue anchor;
allowing the plications to heal;
threading a loop suture through the first and second loops;
drawing the first and second plications together to pulling on the loop suture; and
cinching or knotting the loop suture to hold the plications in the drawn together position.

21-25. (canceled)
